(19) United States
(12) Patent Application Publication
(10) Pub. No.: US 2006/0115469 A1
(43) Pub. Date: Jun. 1, 2006

(54) Interstitial collagenase molecular motor

(75) Inventors: Gregory Goldberg, St. Louis, MO (US); Saveez Saffarian, Cambridge, MA (US); Ivan E. Collier, St. Louis, MO (US); Barry L. Marmer, St. Louis, MO (US); Elliott L. Elson, St. Louis, MO (US)

Correspondence Address: Michael G. Douglas, Ph.D.,;Office of Technology Management, Washington University, Campus Box 8013, 660 S. Euclid Ave, St. Louis, MO, 63110, US

(73) Assignee: WASHINGTON UNIVERSITY, ST. LOUIS, MO (US)

(21) Appl. No.: 11/001,108

(22) Filed: Dec. 1, 2004

(51) Int. Cl. A61K 38/48; C12N 9/64

(52) U.S. Cl. 424/94.63; 435/226

(57) ABSTRACT

A novel interstitial collagenase is disclosed which acts as an ATP-independent molecular motor driven by the proteolysis of substrate collagen. Also disclosed is a method of reacting interstitial collagenase with a collagen fibril to encounter multiple cleavage sites without substantial dissociation of said interstitial collagenase and thereby act as a molecular motor driven by the proteolysis of substrate collagen. The invention thus is useful as a research tool in the tissue remodeling and cell-matrix interaction. The invention also has significant utility in the screening and development of inhibitors of interstitial collagenase and as a research tool for evaluating the activity and effect of drug candidates.

This invention was made in part with government support under grant numbers GM-38838, AR40618 and AR 39472, awarded by the National Institutes of Health. The government has certain rights to the invention.

### FIELD OF THE INVENTION

The present invention relates to the field of interstitial collagenase

### BACKGROUND OF THE INVENTION

The extracellular matrix (ECM) of vertebrates is a three-dimensional scaffold consisting of highly organized macromolecular assemblies that vary in structure and composition to define and maintain the shapes and mechanical properties of tissues. Many physiological and pathophysiological processes from morphogenesis to wound healing, tumor progression and metastatic invasion are characterized by intensified tissue remodeling that begins with degradation of the existing ECM (refs. 1, 2, below).

Collagen is the most abundant component of the ECM. Monomers of fibrillar collagens have a unique triple-helical structure that self assembles to produce tightly packed periodic fibrils (ref. 3) up to 500 nm in diameter that are highly resistant to proteolytic degradation. Resident cells of tissues can secrete a specialized group of enzymes, matrix metalloproteases (MMPs) that degrade ECM macromolecules including collagens (ref. 4). In humans, interstitial collagenase (MMP-1), (ref. 5) is principally responsible for fibrillar collagen turnover. The enzyme cleaves all three a chains of the collagen monomer at a single site located approximately three-fourths of the way from its NH₂ terminus (ref. 6). The assembled collagen fibril contains multiple equidistantly distributed cleavage sites 300 nm apart.

Feynman showed (ref. 7) that particles diffusing in an anisotropic environment cannot produce work in an isothermal system but a thermal gradient applied to the same system can bias the diffusion. This illustration was of considerable importance pointing to the plausibility of microscopic machines (ref. 8). Harnessing work from Brownian motion has been both an exciting and a controversial topic (refs. 9, 10). In the last decade theoretical physicists have produced several models of biased diffusion without a need for a system-wide gradient or a field. For instance, coupling to external fluctuations can create machines known as “thermal ratchets” (refs. 11, 12) that harvest the energy from colored noise (ref. 13). A “Brownian Ratchet” can be powered by coupling to a non-equilibrium chemical reaction driving the particle between two states (refs. 14-16). Recently a “Burnt Bridge” model of a Brownian ratchet has been described (ref. 17). In this model the diffusion bias is created because a moving particle can destroy weak places on a track in a way that inhibits its ability to diffuse back.

### BRIEF DESCRIPTION OF THE INVENTION

The present invention relates to a novel interstitial collagenase molecular motor and method of use operating extracellularly. More particular, the invention comprises an interstitial collagenase (MMP-1) acting as an ATP-independent molecular motor driven by the proteolysis of substrate collagen.

As used herein, MMP-1 is the state-of-the-art enzyme nomenclature for interstitial collagenase. MMP-1 is known to degrade fibrillar collagen (types I, II, and III) but not collagen types IV and V. The complete cDNA and primary structure of interstitial collagenase from human skin fibroblasts is described in U.S. Pat. No. 4,772,557 (see also ref. 5).

It is demonstrated in accordance with the invention that the digestion of a collagen fibril occurs when the bound MMP-1 undergoes biased diffusion along the fibril encountering cleavage sites without noticeable dissociation. The MMP-1 transport mechanism is akin to a Brownian ratchet with biased diffusion independent of ATP hydrolysis but coupled to collagen proteolysis instead.

MMP-1 as thus described herein is believed to be the first example of an ATP-independent extracellular molecular motor. The disclosure herein of MMP-1 acting as a molecular ratchet tethered to the cell surface of a collagen fibril supports its use as a research tool in the tissue remodeling and cell-matrix interaction.

MMP-1 has been known for many years to be useful in the treatment of hypertrophic scars, keloids and intervertebral disc disease. It is implicated in conditions in which degeneration of connective tissue is an important part of the pathology of normal repair and damage. MMP-1 is also recognized as a drug target for the treatment of inflammation, would healing and cancer. Accordingly, the invention as described and claimed herein has significant utility in the screening and development of inhibitors of MMP-1 and as a research tool for evaluating the activity and effect of drug candidates.

In various illustrative examples of the invention hereinafter, Monte Carlo simulations using a model similar to the “burnt bridge” Brownian ratchet were found to accurately describe the actual experimental results on the kinetics of collagen digestion.

The interstitial collagenase molecular motor as disclosed herein also is useful in the field of nanotechnology whereby the molecular motor can manipulate molecules one at a time. This is illustrated herein by Fluorescence Correlation Spectroscopy (FCS) in which an individual MMP-1 decorated collagen fibril represents single molecules of the enzyme passing through the laser beam. The interstitial collagenase can thus act as an ATP-independent motor for cargo delivery in nanotechnology.

The interstitial collagenase acting as an ATP-independent extracellular molecular motor as disclosed herein is further useful in the field of drug delivery whereby attached drug molecules can be transported by the molecular motor.

### DETAILED DESCRIPTION OF THE INVENTION

While the specification concludes with claims particularly pointing out and distinctly claiming the subject matter regarded as forming the invention, it is believed that the invention will be better understood from the following preferred embodiments taken in conjunction with the accompanying drawings.

### BRIEF DESCRIPTION OF THE DRAWINGS

FIG. 1. Mobility of MMP-1 Bound to a Collagen Fibril, Shown in 3 Parts, FIGS. 1A, 1B, and 1C.

The nano-positioning stage was instructed to center a selected individual collagen fibril under the laser beam. A constant average fluorescence indicating a steady state was achieved after the initial exposure of the fibril spot to a 5 mw laser beam for 120 sec. A primary fluorescent signal was collected for 300 sec at 400 μsec intervals immediately following the initial exposure

FIG. 1A. Primary fluorescent intensity record of a MMP-1 decorated (Red) and an untreated collagen fibril (Black) recorded at 400 μsec intervals. Both records are dominated by shot noise.

FIG. 1B. Primary fluorescent intensity record shown in FIG. 1A with the time interval increased to 80 msec to reduce the shot noise. Spikes of fluorescence intensity are observed in the experiments performed on MMP-1 decorated fibrils. Background fluorescence from untreated collagen fibrils shows no spikes in the record.

FIG. 1C. The data at 80 msec time resolution in FIG. 1B were used for finding the position of the spikes in the fluorescence record using a threshold method with the threshold set to 5 times the standard deviation calculated from the average signal in each of the 2 sec windows. Isolated from the background, the spikes of fluorescence intensity represent single molecules of MMP-1 passing through the laser beam.

The single molecule fluorescence was measured in FCS experiments with MMP-1 in solution. The average value of 870 Hz (Black line) and a maximum of 2.4 KHz (Gray line) were calculated by either averaging the fluorescence over the entire observation volume or assuming the molecule traveled through the center of the beam respectively. The intensity of spikes is within the expected range for a single molecule intensity.

FIG. 2. Activated MMP-1 is a ATP-Independent Motor Enzyme Driven by Proteolysis of its Substrate, Collagen.

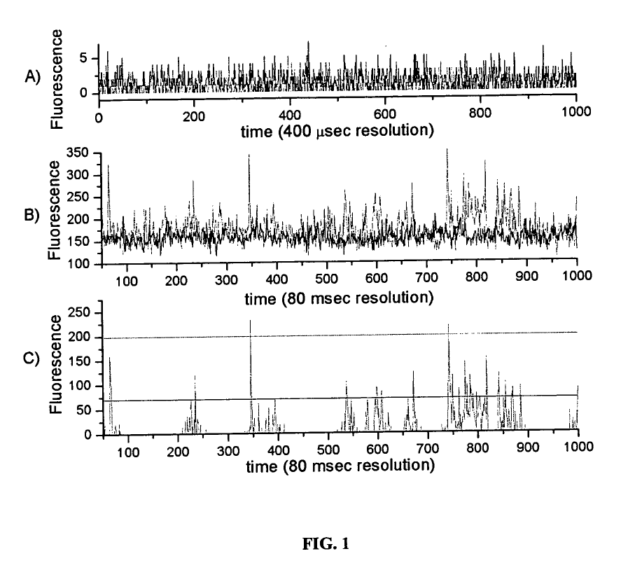

Experimental correlation functions for the spikes of fluorescence presented in the FIG. 1C were calculated using 400 μsec time resolution data obtained from collagen fibrils decorated with activated wild type MMP-1 (Red) or its inactive mutant (Black). The experimental correlation function for wild-type (WT) activated enzyme was fitted (Gray line) to a flow plus diffusion model (20),

$$G ( t ) = Exp [ - {( \frac{t}{{τ}_{f}} )}^{2} \frac{1}{1 + \frac{t}{{τ}_{D}}} ] \frac{1}{{( 1 + \frac{t}{{τ}_{D}} )}^{0.5}} , where$$

$${τ}_{D} = \frac{{w}^{2}}{4 D} is the diffusion time , \\ {τ}_{F} = \frac{w}{V} is the flow time ,$$

w is the beam waist, D is the diffusion coefficient, and V is the flow velocity.
The shown fit of the WT MMP-1 data has a local diffusion coefficient of 8±1.5×10⁻⁹ cm² sec⁻¹ and a transport velocity of 4.5±0.36 μm sec⁻¹.
The correlation function of the inactive mutant exhibits a long tail characteristic of an unbiased 1-D diffusion

$$G ( τ ) = \frac{1}{{( 1 + \frac{t}{{τ}_{D}} )}^{0.5}} ,$$

with a local diffusion coefficient of 6.7±1.5×10⁻⁹ cm² sec⁻¹.
FIG. 3. The Unequal Flux of MMP-1 Molecules Around a “No Transport” Block on the Collagen Fibril.

Photo-bleaching of MMP-1 decorated collagen fibrils with a laser beam intensity below 50 mw results in the recovery of fluorescence after termination of exposure. Raising the laser intensity to 80-90 mw prevents the recovery after photo-bleaching indicating a blockage of the enzyme transport across the bleached area due to a damage of a fibril.

The average number of single enzyme molecules passing through the laser beam in the left (C_{L}) and right (C_{R}) flanks of the “no transport” block are calculated from three independent experiments for each form of the enzyme.

The differences are expressed as the asymmetry ratio calculated as

$$\frac{{C}_{L} - {C}_{R}}{{C}_{L} + {C}_{R}}$$

so that 1 indicates a perfect asymmetry and 0 a completely symmetric experiment. The arrows indicate the direction of enzyme transport.
FIG. 4. “Burnt Bridges” Mechanism of MMP-1 Transport on a Collagen Fibril, Shown in 2 Parts, FIGS. 4A and 4B.
FIG. 4A. The Rules of the Road.

The enzyme molecules perform a random walk in one-dimension along a collagen fibril. Once they reach a cleavage recognition site, a successful cleavage occurs with a set probability P_{J} and the enzyme molecule responsible for the cleavage will always end up on one side of the cleaved peptide bond. The molecules are not allowed to cross the cleaved triple helix from either side but are allowed to jump to a neighboring triple helix track with a small probability P_{J} This mechanism, akin to a “Brownian ratchet”, produces a net transport with velocity V which depends on a diffusion coefficient, the probabilities defined above and a spatial distribution of the cleavage sites.

FIG. 4B. Monte Carlo Simulations of the Interaction of MMP-1 with a Collagen Fibril.

To construct a Monte Carlo simulation of MMP-1 motion on collagen, 100 enzyme molecules on a 30 μm long fibril with a cross-section of 100 triple helixes of collagen and cleavage sites 0.3 μm apart were monitored. In the beginning of an experiment MMP-1 molecules were positioned randomly on a fibril and were allowed to diffuse according to the rules described above. A value of P_{J} was set at 1/200 sec. Once molecules exited the monitored 30 μm region they are put back on the opposite end of the fibril to approximate the conditions on a long fibril. The size of random walk steps has a Gaussian distribution as shown. Walkers encounter a reflective boundary condition at each of the “burnt bridges”.

FIG. 5. Monte Carlo Simulations of the Experimental Correlation Functions and Concentration Profile of MMP-1 on Collagen Fibril, Shown in 2 Parts, FIGS. 5A and 5B.

FIG. 5A. Two simulated correlation functions for the P_{C}=10% (Red) and P_{C}=0 (Black) were fitted as in FIG. 3 with diffusion plus flow and 1D diffusion models respectively. They are in good agreement with experimental functions obtained for WT and inactive mutant enzymes.

FIG. 5B. The simulated concentration profiles of WT (Red) and inactive mutant (Black) enzymes on a collagen fibril. The simulation was composed by monitoring 100 molecules on a 30 μm long fibril with a cross-section of 100 triple helices and proteolytic sites spaced at 0.3 μm producing an average occupation number of 1 for each of the 0.3 μm segments. The concentration profile was summed over 24 sec of the simulation.

FIG. 6. Monte Carlo Simulation of the Flux of Single MMP-1 Molecules Around a “No Transport” Block on a Collagen Fibril.

Monte Carlo simulations were performed as in the FIG. 5B with a reflective boundary condition set in the middle of the fibril representing the “no transport” block. The number of molecules on each side of the block was recorded to calculate the asymmetry ratios as in the FIG. 4. The simulations demonstrate that the asymmetry ratio is highly dependent on the value of P_{C}. The asymmetry ratios obtained experimentally with active MMP-1, its inactive mutant and MMP-1 inhibited by the protease inhibitor, Galadrin or heavy water are indicated.

In order to further illustrate the invention in greater detail, the following specific Examples are provided. Although specific Examples are thus illustrated herein, it will be appreciated and understood that the invention is not limited to these specific Examples or the details therein.

For convenience and reference, various scientific publications on conventional materials, laboratory procedures, and other state-of-the-art techniques and procedures commonly used and well-known to the person skilled in the art and employed in the following Examples are indicated by reference numerals in parentheses and cited at the end of the specification under the heading “References.”

### EXAMPLES

The specific methods and materials used in the following illustrative Examples are as follows:

Methods and Materials.

Labeling Pro-MMP-1 Enzyme

The human recombinant Interstitial Collagenase (MMP-1) was expressed in mammalian cells and purified from serum free conditioned media. The enzyme was labeled with Alexa 488 fluorescent dye using the Alexa Fluor Protein labeling kit (Molecular Probes, A-10235). One hundred microgram of enzyme was absorbed to a 50 μl bed volume column of Reactive Red −120 Agarose resin (Sigma A-0503, Sigma-Aldrich Chemical Co., St Louis, Mo.) equilibrated with 25 mM HEPES buffer pH=8.0, containing 2 mM CaCl₂ and 50 mM NaCl (Buffer A) and incubated with the Alexa 488 dye for 30 min at RT. The enzyme was eluted with the same buffer containing 2 M NaCl and dialyzed against 25 mM HEPES pH=7.5 buffer containing 2 mM CaCl₂, 150 mM NaCl and 0.005% Brij-35.

Mutant Preparation.

The active center mutant “E219Q” was constructed using PCR site directed mutagenesis and MMP-1 cDNA as a template (ref. 5). The resulting mutant was sub-cloned into expression vector p6RHyg and transfected into p2AHT2a cells for expression (ref. 33). The mutant “E219Q” has been previously characterized (ref. 34) as having a normal binding activity but completely inactive in collagen proteolysis.

Enzyme Activation

Alexa-488 labeled MMP-1 was activated in 25 mM HEPES pH=7.5 buffer containing 2 mM CaCl₂, 150 mM NaCl, and 005% Brij-35 by addition of Plasmin and purified Stromelysin (MMP-3) to achieve 1:5 and 1:25 molar ratios, respectively. The enzyme mixture was incubated for 1 hr at 37° C. and the plasmin activity was inhibited by addition of a 5 fold molar excess of aprotinin. The activation of MMP-1 was visualized on SDS-PAGE as conversion of pro-enzyme to 42 kDa MW enzyme.

Preparation of the Oriented Collagen Gels (ref. 18).

Acid soluble rat tail collagen was neutralized at 4° C. with Tris-HCl pH=7.5 buffer and dispensed into 0.24 mm thick chambers the bottoms of which were made from chemically derivatized glass slides (Silyated slides CSS-25, CEL Associates Inc., Pearland, Tex.). The sealed chambers were placed onto a temperature-controlled copper probe suspended in a perpendicular orientation to the magnetic field of 8 Tesla in a super-conducting magnet (The Laboratory of Magnetism, Department of Physics, Washington University). Polymerization of the gels was initiated by raising the temperature from 10° C. to 37° C. over 4 Hours.

Sample Preparation.

Oriented collagen gels were purged with nitrogen prior to addition of Alexa-488 labeled MMP-1 at a final concentration of 0.2 μM and incubated on ice for 15 min. Gels were washed extensively to achieve a residual concentration of the free enzyme below 10 nM as monitored by FCS in spaces between the fibrils of the gel samples. These FCS data were also used to measure the brightness of the fluorescent enzyme molecules. To inhibit MMP-1 activity Galardin (GM 6001, BioMol Research Laboratories Inc.) was added to a final concentration of 20 μM. In the experiments with heavy water the collagen gels were incubated in excess of heavy water for 24 hours and all the solutions required for sample preparation were made in heavy water. Trolox (Aldrich) was added to the washed gels at 2.5 mM final concentration to inhibit photo-bleaching.

The Fluorescence Correlation Spectroscopy (FCS) Experiments.

The FCS setup consisted of a titanium sapphire laser coupled to an Olympus IX-70 inverted microscope equipped with a p527.3CL piezzo electric stage and e-710.4CL controller (Physik Instrumente, Germany) was described previously (ref. 35), The 5 mwatts intensity laser beam was tuned to 810 nm for all FCS experiments. The scanning or move commands were sent to the stage controller by the home written Labview program. The collagen gel chambers were mounted on top of the piezzo electric stage and a fluorescent image was produced by scanning an area of the gel across the laser beam. A selected individual fibril was scanned with a higher zoom of 100 nm/pixels. A selected experimental spot was centered under the beam and its position was verified by observation of a high fluorescence at the time of initial contact. The spot was exposed for 120 sec to achieve a constant average fluorescence indicating a steady state. The fluorescence intensities were recorded for 300 sec at 400 μsec intervals starting immediately after the initial exposure.

The Spatial Filter.

Reducing the time resolution of the fluorescent record from 400 μsec to 80 msec revealed spikes of intensity present in MMP-1 decorated fibrils that were absent in control. The 80 msec fluorescent record was scanned with a 2 sec window to determine a local fluorescence intensity average. A fluorescent signal exceeding the 5 fold the standard deviation of the local background signal was defined as a spike and its location in time and its average local background were saved. Once the location of the spikes were recorded the 400 μsec data were processed so that the intensities around the spikes were put equal to zero, while the points within the window containing a spike were put to the original intensity minus the average local background contribution. The statistics of the background signal were poissonian. The resulting data were fed to a software correlator to produce a correlation function.

As a control to the validity of the spatial filter a spike profile generated through Monte Carlo simulations of a one dimensional random walker was added to a background intensity record and correlated using the spatial filter. The resulting correlation function was compared with the correlation function of the simulated spikes without addition of the background and the two were found to be in very good agreement. Therefore the threshold method has proved very effective in filtering the background fluctuations without compromising the accuracy of the correlation functions

Monte Carlo Simulations of the Interaction of MMP-1 with a Collagen Fibril.

To construct a Monte Carlo simulation of MMP-1 motion on collagen, monitoring was had of 100 enzyme molecules on a 30 μm long fibril with 100 triple helical monomers cross-section represented as 100 independent tracks with equidistant cleavage sites 0.3 μm apart. In the beginning of an experiment MMP-1 molecules were positioned randomly on a fibril and were allowed to diffuse according to the rules described in FIG. 4. A value of P_{J} representing the probability of a molecule jumping between tracks was set at 1/200 sec. Once the molecules exited the monitored 30 μm region they were put back on the opposite end of the fibril to approximate the conditions on a long fibril. The size of random walk steps had a Gaussian distribution and the walkers encountered a reflective boundary condition at each of the cleaved recognition sites termed “burnt bridges” (ref. 17).

To simulate an FCS experiment, a Gaussian laser beam 300 nm in waist was considered in the simulations. Molecules traveling through the beam produced fluorescence intensity proportional to the laser intensity at their position. Each molecule participating in the simulation was allowed to contribute a maximum number of photons predetermined by a random number following a Gaussian distribution with an average of 500 and a width of 500 photons. After the total number of photons exceeded the maximum the molecule was removed from the simulation. To maintain the total number of the molecules on the fibril a replacement fresh molecule was placed on the fibril at a random location.

In the simulations of the asymmetry ratio measurements, a reflecting boundary is set in the middle of the fibril so that molecules approaching it from either direction were reflected back. In these simulations the number of molecule each side of the boundary were counted and the number was used in calculation of the asymmetry ratios produced by the simulations.

### Example 1

To observe the behavior of single MMP-1 molecules bound to a collagen fibril the oriented collagen gels (ref. 18) produced by polymerizing rat-tail collagen in a magnetic field of 8T were decorated with Alexa 488 labeled enzyme. A selected individual fibril was centered under the 5 mw

laser beam and illuminated for 120 sec to achieve a constant average fluorescence, indicating a steady state. The initial exposure was immediately followed by collection of primary fluorescence data for 300 sec at 400 μsec time intervals (FIG. 1A).

The control record was obtained from the undecorated fibrils using collagen luminescence for imaging. The primary fluorescence data from both the experiment and the control were dominated by shot noise. Decreasing the time resolution of the primary record from 400 μsec to 80 msec (FIG. 1B) revealed large spikes of fluorescence in the record obtained from MMP-1 decorated fibrils that were absent in the control record. Since the single molecules of Alexa 488 labeled MMP-1 are much brighter than the background, the presence of the spikes can be explained by passage of enzyme molecules through the laser beam. To isolate the spikes from the background, the 80 msec time resolution data (FIG. 1B) was filtered using a threshold method (described above under “The Spatial Filter”) with the threshold set to 5 times the standard deviation calculated from the average signal in each of the 2 sec windows (FIG. 1C).

The intensity of the spikes observed on MMP-1 decorated fibrils was well within the limits of the brightness expected of a single Alexa 488 labeled MMP-1 molecule measured in solution FCS experiments where the average MMP-1 molecule traverse the beam in 120 μsec [FIG. 1C, (see “The Spatial Filter”)]. It is thus concluded that the spikes of fluorescence intensity observed in FCS experiments on an individual MMP-1 decorated fibril represent single molecules of the enzyme passing through the laser beam.

### Example 2

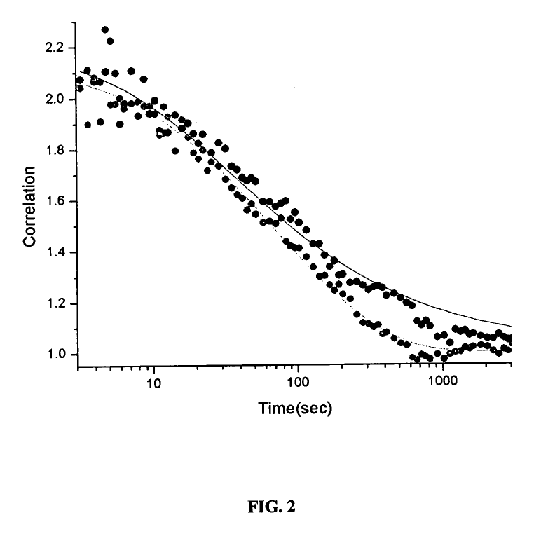

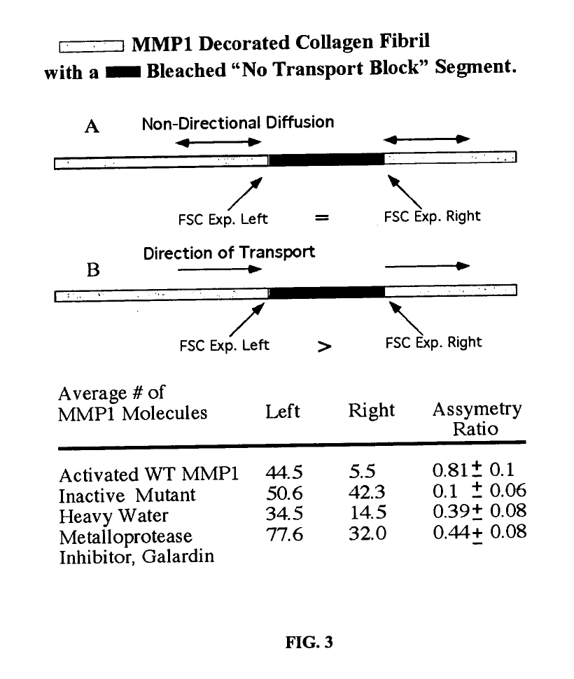

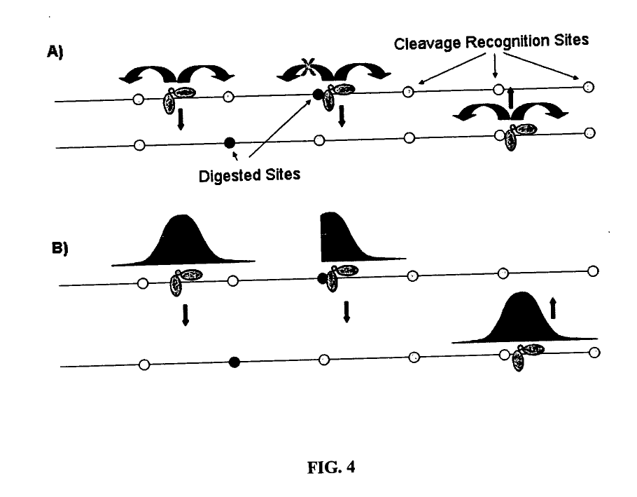

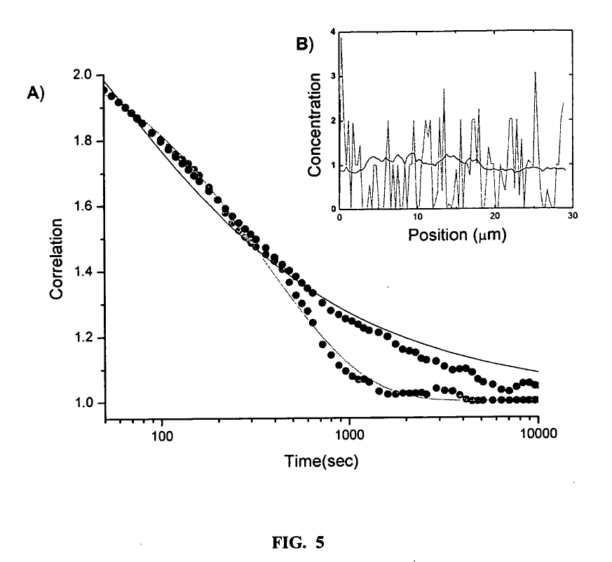

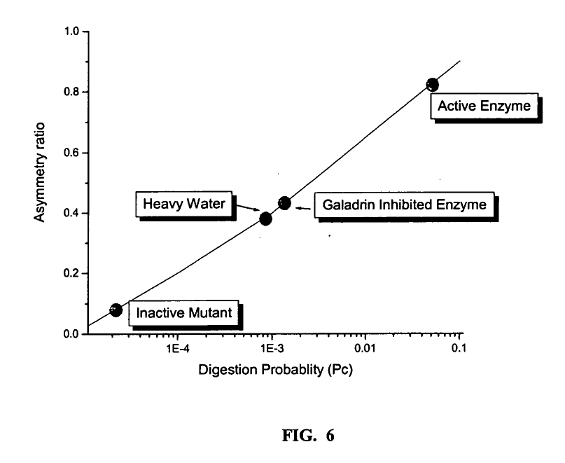

Next the isolated fluorescence record of the spikes of Example 1 was subjected to correlation function analysis (FIG. 2). A particle diffusing freely in one dimension has a finite probability of returning to a point that it visited previously. Therefore the correlation function characteristic for 1-D diffusion contains an elongated tail. The experimental correlation function for the active enzyme (FIG. 2) lacks this feature and thus fits well into a 1-D diffusion plus flow model (see “The Spatial Filter”) in which particles exhibit diffusive behavior at fast time scales but have zero probability of return at longer times due to the directional flow.

The local diffusion coefficient D=8±1.5×10⁻⁹ cm² sec⁻¹ and the transport velocity V=4.5±0.36 μm sec⁻¹ were determined from the fit of the correlation function obtained from the wild type activated MMP-1. Biased diffusion is a characteristic of a molecular motor and requires energy dissipation. In the classical molecular motors the required energy is supplied by hydrolysis of ATP (refs. 20-22). In the absence of ATP it was hypothesized that the proteolysis of collagen monomers catalyzed by the active enzyme is a possible source of energy.

To investigate this possibility, the enzyme was inactivated by a single point mutation, E219Q in its active center (see “The Spatial Filter”). FCS measurements on the inactive MMP-1 mutant produced experimental correlation functions that exhibited an elongated tail, the signature characteristic of the 1-D diffusion (FIG. 2). The diffusion coefficient D=6.7±1.5×10⁻⁹ cm² sec⁻¹ recovered from the correlation functions of the inactive mutant was strikingly similar to the diffusion coefficient of the active enzyme. The similarity of diffusion coefficients argues that the inactivation of the enzyme has little effect on the diffusion properties of MMP-1 on the collagen fibrils and that the transport mechanism is dependent on the catalytic activity of the enzyme.

### Example 3

The above FCS experiments of Example 2 investigated the properties of MMP-1 transport near a microscopically small observation volume. To verify these results and to determine whether the bias component dominates the transport process on a macroscopic scale, the flux of single MMP-1 molecules around a “no transport” block created on a collagen fibril (FIG. 3) was measured. Photo bleaching of MMP-1-decorated collagen fibrils with a laser beam intensity below 50 mw is followed by a recovery of fluorescence after termination of the bleach pulse. Raising the laser intensity to 80-90 mw prevents the fluorescence recovery, indicating a blockage of the enzyme transport across the bleached area due to damage to the fibril. The average number of single enzyme molecules passing through the laser beam near the left (CL) and the right (CR) flanks of the “no transport” block would be equal for unbiased diffusion. A flux difference between the upstream and downstream regions would indicate directional transport of the MMP-1 on the fibril (FIG. 3).

The number of fluorescent spikes observed in the FCS experiments on each side of the “no transport” block was used to measure the flux. An asymmetry ratio is defined as the difference between the fluxes on the opposite sides of the block divided by the total flux of the molecules so that a value of 1 indicates perfect asymmetry (flow dominates diffusion) and a value of 0, a completely symmetric diffusion. Results presented in FIG. 3 demonstrate that the flux is highly asymmetric for the wild type active enzyme while the inactive mutant shows complete symmetry. Intermediate results were observed when proteolysis is inhibited in the presence of the metalloprotease inhibitor, Galardin, or by immersion of the collagen fibrils in heavy water (ref. 23). These results support the conclusion that the active MMP-1 bound to a collagen fibril undergoes proteolysis-dependant directional transport and further demonstrate that the degree of asymmetry in the flux depends on the efficiency of proteolysis.

### Example 4

The following picture of the interaction of MMP-1 with a collagen fibril emerges. When the proteolytic activity of MMP-1 is inhibited, the enzyme molecules perform a random walk in one-dimension along the fibril, characterized by the diffusion coefficient determined from FCS experiments. The proteolytically active enzyme is capable of directional transport with a local velocity V=4.5±0.36 μm sec⁻¹ also determined experimentally. This Example 4 shows the mechanism that rectifies symmetric diffusive motion to induce biased transport of the active enzyme along the fibril.

A theoretical model similar to a recently formalized “Bumt Bridges” Brownian Ratchet (ref. 17) was considered to see if the experimental results can be explained based on the following two assumptions (FIG. 5):i. Once an enzyme walker encounters a cleavage recognition site, a successful cleavage occurs with a probability Pc and the enzyme molecule responsible for the cleavage always ends up on a specific side of the cleaved peptide bond; ii. The molecules are not allowed to cross the cleaved collagen helix from either side but are allowed to jump to a neighboring triple helix track with a small probability Pj. This mechanism based on random walks produces a net transport (ref. 17) with velocity V that depends on a diffusion coefficient, the probabilities defined above and the spatial distribution of the cleavage sites along the tracks (300 nm apart for a collagen fibril). The analytical solution of this model for one molecule on a single track with the equidistant distribution of sites and low Pc is given in reference 17. An analytical solution for an ensemble of enzyme molecules traveling on the same track is not available because of their interaction.

To model the behavior of an ensemble of the enzyme molecules diffusing on a fibril, Monte Carlo simulations were constructed monitoring 100 enzyme molecules walking under the rules set above. A 30-micron long collagen fibril composed of 100 triple helices in cross-section with recycling boundary conditions (see “The Spatial Filter”) was considered. Both correlation functions and asymmetry ratios can be obtained from the simulations for a set value of Pc and Pj. The simulated correlation functions (FIG. 5) reproduced those obtained experimentally (FIG. 1) when the probability of successful cleavage upon encounter Pc=10%, the rate of jumping to a neighboring triple helix track Pj= 1/200 sec⁻¹ and the diffusion coefficient D=8±1.5×10⁻⁹ cm² sec⁻¹ measured experimentally. Simulations using the same set of parameters accurately predict the asymmetry ratio measured experimentally for the active enzyme (FIG. 6) while setting the value of Pc to 2×10⁻⁵ predicted the asymmetry ratio observed for the mutant enzyme. Simulations also demonstrate that the asymmetry ratio has an exponential dependence on the probability of cleavage Pc so that a 50% decline in the asymmetry ratio corresponds to a two orders of magnitudes drop in the cleavage probability. Thus Monte Carlo simulations of the correlation functions and the asymmetry ratios accurately predict the experimental results for both active and inhibited enzymes depending on the value of a single parameter Pc. This is a notable observation since the measurements of correlation functions and the asymmetry ratios are experimentally independent.

The model described herein entails a large scale interaction of the enzyme molecules diffusing on a fibril since cleavage of a triple helical track by a passing enzyme acts as a road block for all the following molecules traveling on the same track. With time this translates into the accumulation of enzyme molecules that are restricted to free diffusion between neighboring cleavage sites 300 nm apart, creating a traffic jam. At room temperature the dissociation of the digested monomer from the surface of the fibril is slow so that the traffic jam can be observed experimentally as a large portion of the fluorescent signal bleached within the first 120 seconds of beam exposure prior to establishment of a steady state.

The traffic jam phenomena explain an early study of the kinetics of collagen digestion in which MMP-1 exhibited simultaneous substrate and enzyme saturations (ref. 24). In these experiments the overall speed of collagen digestion increased linearly with the enzyme concentration. At a nonsaturating concentration of collagenase and 17-fold molar excess of collagen substrate the enzyme activity rose significantly as the amount of substrate was further increased three fold. Under the traffic jam conditions, presenting more collagen to the enzyme would reduce the average concentration of the enzyme molecules on the tracks thus relieving the traffic jam and increasing the overall speed of digestion. Thermodynamic measurements further support this interpretation. The activation energy for fibril digestion (ref. 23) was found to be much larger (101 kcal mol⁻¹) than that of most other enzymes including proteases. The high energy of enzyme activation was associated with the highly polymerized state of collagen fibrils since the activation energy for digestion of the triple helical collagen monomer was 4 times lower (ref. 23). The high energy of activation for collagenolysis is in good agreement, however, with the apparent energy of activation for collagen fibril unfolding (124 kcal mol⁻¹) measured more recently (ref. 25). Although the activation energy for the dissociation of a cleaved monomer is expected to be lower than that of an intact collagen fibril, these results are consistent with the idea that at certain temperatures the rate-limiting step of collagen fibril digestion is the dissociation of the cleaved monomers from the surface of the fibril.

In the computer simulations a partial relief of the traffic jam is accomplished by letting the enzyme walker acquire a new track with the probability PJ. When the track jumping rate is slow (PJ= 1/200 sec⁻¹) and PC=10% most of the active enzyme molecules eventually become trapped between the two neighboring cleavage sites creating spikes of the enzyme concentration along the fibril. Under these conditions setting the P_{C}=0 is sufficient to achieve an even distribution of the enzyme along the fibril (FIG. 5B, insert).

It is thus concluded that the MMP-1—collagen system is the first example of a new class of ATP-independent molecular motors operating extracellularly. The mechanism of this motor is akin to a Brownian ratchet that is able to rectify Brownian forces into a propulsion mechanism by coupling to an energy source, in this case collagen proteolysis. Further studies can determine the efficiency of energy coupling in this system. The upper limit of the energy density of the MMP-1 motor can be calculated from the energy of peptide bond cleavage to be 1.4×10⁻¹⁸ Watts/molecule of MMP-1. The absolute lower limit, 1.7×10⁻²⁰ Watts/molecule of MMP-1, can be defined by the energy required to propel an MMP-1 molecule through an aqueous solution with the velocity V=4.5±0.36 μm sec⁻¹.

Further investigation may elucidate additional characteristic details of the interaction of the MMP-1 with collagen. The enzyme is likely to contain at least two collagen binding sites to actuate the processive diffusion. Both deletion and domain exchange studies (ref. 26) suggest that in addition to the active center, the C-terminal domain of collagenases plays an important role in substrate interaction. The specific activities of collagenases and their C-terminal truncated forms were found to be similar using peptide substrates, while truncated collagenases failed to cleave native triple helical collagens (refs. 27, 28). Furthermore, mutations in the flexible hinge region connecting the catalytic and the C-terminal domains caused a 99% drop in collagenolytic activity (ref. 28) suggesting the importance of their relative orientation. Finally, recent observations (ref. 29) clearly demonstrate that the C-terminal domain of a closely related enzyme, MMP-2, is required for its diffusion on a layer of gelatin substrate.

The biological consequences of the collagenase motor activity are of significant interest. It is believed that the collagenase ratchet can serve as a clutch mechanism assisting cell locomotion on collagen matrices and contraction of collagen gels in three-dimensional cultures. Association of the enzyme with cell membranes via interaction with tissue-specific integrins can couple the extracellular proteolysis with the forces exerted by the cytoskeleton to direct membrane protrusions along a “no skid” surface generated by the digestion of collagen fibrils. Membrane-type MMPs (refs. 30, 31) can potentially act in a similar fashion. Experimental observations that clearly demonstrate the requirement of MMP-1-dependent collagenolysis for migration of keratinocytes on collagen support this hypothesis (ref. 32).

Although illustrative examples of the invention are provided herein, it will be understood that the invention is not limited to these specific examples but includes such other examples as will be apparent to the person skilled in the art after reading the disclosure herein.

### SCIENTIFIC REFERENCES

1. T. Vu, W. Z, Genes Dev. 14, 2123-33 (2000).
2. M. Egeblad, W. Z., Nat Rev Cancer 2, 161-74 (2002).
3. K. E. Kadler, J. A. T. J. A. C. David F. Holmes, Biochem. J. 316, 1-11 (1996).
4. R. Visse, N. H., Circ Res. 92, 827-39 (2003).
5. G. I. Goldberg et al., J. Biol. Chem. 261, 6600-5 (1986).
6. G. Fields, V. W. HE, B.-H. H., J Biol Chem. 262, 6221-6 (1987).
7. R. P. Feynman, R. B. Leighton, M. Sands, The Feynman Lectures on Physics (Addison-Wesley, 1966), vol. 1.
8. R. D. Astumian, Science 276, 917-922 (1997).
9. R. Astumian, Sci Am. 285, 56-64 (2001).
10. R. Astumian, Philos Trans R Soc Lond B Biol Sci. 355, 511-22 (2000).
10. R. D. Astumian, P. B. Chock, T. Y. Tsong, Y. D. Chen, H. V. Westerhoff, Proc. Natl. Acad. Sci. U.S.A. 84, 434 (1987).
12. M. O. Magnasco, Phys. Rev. Lett. 71, 1477-1481 (1993).
13. A. A. Frank Jülicher & Jacques Prost, Rev. Mod. Phys. 69, 1269-1282 (1997).
14. C. Peskin, G. Odell, G. Oster, Biophys. J. 65, 316-324 (1993).
15. J. Prost, J. F. Chauwin, L. Peliti, A. Ajdari, Phys. Rev. Lett. 72, 2652-2655 (1994).
16. R. F. Fox, Phys. Rev. E 57, 2177-2203 (1998).
17. J. Mai, I. M. Sokolov, A. Blumen, Physical review E 64, 011102 (2000).
18. J. Torbet, R. M C., Biochem J. 219, 1057-9 (1984).
19. D. Magde, E. L. Elson, and W. W. Webb, Biopolymers 17, 361-76 (1978).
20. M. Schliwa, G. Woehike, Nature 422, 759-765 (2003).
21. R. D. Vale, R. A. Milligan, Science 288, 88-95 (2000).
22. H. Qian, Biophysical Chemistry 83, 35-43 (2000).
22. J. J. Jeffrey, H. Welgus, R. Burgeson, A. Eisen, J. Biol. Chem.258,11123-7 (1983)
23. H. Welgus, J. Jeffrey, G. Stricklin, W. Roswit, A. Eisen, J. Biol. Chem. 255, 6806-6813 (1980).
24. E. Leikina, M. V. Mertts, N. Kuznetsova, S. Leikin, Proc. Natl. Acad. Sci. U.S.A. 99, 1314-8 (2002).
25. G. Murphy et al., J. Biol. Chem. 267, 9612-8 (1992).
26. J. L. Lauer-Fields, K. A. Tuzinski, K. Shimokawa, H. Nagase, and G. B. Fields, J. Biol. Chem. 275, 13282-90 (2000).
28. V. Knauper et al., J. Biol. Chem. 272, 7608-16 (1997).
27. I. E. Collier, S. Saffarian, B. L. Marmer, E. L. Elson, G. Goldberg, Biophysical Journal 81, 2370-2377 (2001).
30. T. Shimada, O. E. Nakamura H, Fujii Y, Murakami Y, Sato H, Seiki M, O. Y. Eur J Biochem. 262, 907-14 (1999).
31. E. Ohuchi, F. Y. Imai K, Sato H, Seiki M, Okada Y., J Biol. Chem. 272, 2446-51 (1997).
32. B. K. Pilcher et al., J. Cell Biol. 137, 1445-57 (1997).
33. G. I. Goldberg, A. Strongin, J. Biol. Chem. 267, 4583-91 (1992).
34. D. Steele et al., Protein Eng. 13, 397-405 (2000).
34. S. Saffarian, E. L. Elson, Biophysical Journal 84, 2030-2042 (2003).

What is claimed is:

1. Interstitial collagenase acting as an ATP-independent molecular motor driven by the proteolysis of substrate collagen.

2. A method of reacting interstitial collagenase with a collagen fibril to produce biased diffusion along the surface of said collagen fibril to encounter multiple cleavage sites without substantial dissociation of said interstitial collagenase and thereby act as an ATP-independent molecular motor driven by the proteolysis of substrate collagen.

3. Interstitial collagenase acting as an ATP-independent molecular motor driven by the proteolysis of substrate collagen for cargo delivery of molecules one at a time.

4. A method of drug delivery whereby attached drug molecules are transported to a treatment site by interstitial collagenase acting as an ATP-independent extracellular molecular motor.
